(19) United States
(12) Patent Application Publication
(10) Pub. No.: US 2006/0216816 A1
(43) Pub. Date: Sep. 28, 2006

(54) Flow path system provided with reaction section suited for the detection of hybridization, and hybridization detecting device making use of the flow path system

(76) Inventor: Michihiro Ohnishi, Kanagawa (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/378,361

(22) Filed: Mar. 20, 2006

(30) Foreign Application Priority Data
Mar. 22, 2005 (JP) P2005-081033

(51) Int. Cl. C12M 1/34

(52) U.S. Cl. 435/287.2; 435/288.7

(57) ABSTRACT

A flow path system suitable for use in the detection of hybridization includes a capillary provided at a predetermined location thereof with a reaction section packed with beads. On the beads, a linker having a sequence of bases of the same type is immobilized, and a target nucleic acid complementarily bound to the sequence of the bases of the same type is held.

### CROSS REFERENCES TO RELATED APPLICATIONS

The present invention contains subject matter related to Japanese Patent Application JP 2005-081033 filed in the Japanese Patent Office on Mar. 22, 2005, the entire contents of which being incorporated herein by reference.

### BACKGROUND OF THE INVENTION

This invention relates to a technology for the detection of hybridization. More specifically, this invention is concerned with a technique for allowing hybridization to proceed in a predetermined reaction section arranged in a flow path system and detecting it.

In recent years, integrated bioassay plates holding thereon predetermined DNAs microarrayed by microarray technologies and generally called “DNA chips” or “DNA microarrays” (hereinafter collectively called “DNA chips”) have been developed, and are finding utility in gene mutation analyses, SNPs (single-base polymorphisms) analyses, gene expression frequency analyses, gene network analyses, and the like. In addition, they are expected to find broad applications in drug developments, clinical diagnoses, pharmacogenomics, tailor-made remedies, research on evolution, forensic medicine, and other fields.

Sensor chip technologies represented by such DNA chips and protein chips with proteins integrated thereon quantitate the existing amounts of target substances by making use of specific interactions between detecting substances (which are often called “probes”) immobilized on solid-phase plates and the target substances.

Taking a DNA chip as an example, single-stranded DNA fragments having a segment of the DNA sequence of a target to be analyzed are immobilized beforehand. If DNA molecules having a sequence complementary to the DNA fragments exist in a sample, the DNA fragments and the DNA molecules specifically combine together (in other words, hybridize with each other) to form double-stranded DNA. Relying upon the detection of this double-stranded DNA by a fluorescence labeling technique or the like, a determination is made as to whether or not the DNA molecules have been expressed in the sample solution. Immobilization of numerous DNA fragments of different DNA sequences makes it possible to efficiently perform an analysis as to whether or not plural kinds of DNAs have been expressed or to provide an analysis of expression of a single kind of DNA with redundancy such that the accuracy of the analysis is increased.

Further, techniques which make use of a flow path and a capillary in DNA chips or the like have been proposed recently. For example, a technique is proposed in Japanese Patent Laid-Open No. 2005-030906, which makes it possible to reduce to a small level the amount of each liquid sample required for its analysis. Another technique is proposed in Japanese Patent Laid-Open No. Hei 10-170427, which forms an optical detection unit at a periphery of a capillary to use the capillary as a cell for detection. A further technique is proposed in Japanese Patent Laid-Open No. Hei 06-094722, which allows an interaction to proceed in a capillary path and detects the resulting flow characteristics. These related art techniques are referred to herein for their disclosure of general techniques that use narrow flow paths in the detection of interactions between substances, although they are not specifically relevant to the present invention.

### SUMMARY OF THE INVENTION

Devices in related art, such as DNA chips, or hybridization detecting systems of the construction that a site of reaction is formed on a plate and an oligonucleotide is used as a probe nucleic acid in the reaction site, the spatial volume of the reaction site arranged on the plate is relatively large. These devices or hybridization detecting systems in related art are, therefore, accompanied by a technical problem in that the efficiency of hybridization which proceeds relying upon natural Brownian motion is low and the time required for the hybridization becomes longer. They also involve another technical problem in that their repeated use is hardly feasible for stains or the like on plates.

The present inventors has, therefore, recognized the provision of a novel technique for the detection of hybridization, which assures a good reaction efficiency and permits repeated use as primary.

The present invention provides a flow path system including a capillary provided at a predetermined location thereof with a reaction section packed with beads, wherein on the beads, a linker having a sequence of bases of the same type is immobilized, and a target nucleic acid complimentarily bound to the sequence of the bases of the same type is held, and further, a multiple flow path system including a plurality of flow path systems as defined above.

It is to be noted that the term “sequence of bases” or “base sequence” as used herein means two or more bases polymerized together. The term “linker” as used herein means a nucleic acid of a predetermined sequence useful for holding a target nucleic acid on beads. Further, the term “target nucleic acid” as used herein means a nucleic acid, which is standing by in a reaction section for the confirmation of the formation of a complementary chain with a probe nucleic acid and is held on the beads via the linkers.

The “reaction section” arranged in the flow path system functions as a site, for example, for allowing hybridization to proceed between a single-stranded segment of the target nucleic acid and a probe nucleic acid fed into the reaction section.

It is also to be noted that the term “probe nucleic acid” as used herein means a nucleic acid which can provide useful information for the detection of hybridization and can be, for example, a single-stranded nucleic acid labeled with a fluorescent substance, a single-stranded nucleic acid labeled with a radioactive substance or the like. Hybridization can be determined by detecting excited fluorescence in the former or a radiation in the latter.

The sequence of the bases of the same type in the linker can be, for example, a polyT, and the probe nucleic acid can be, for example, a mRNA having a polyTtail fragment which is complimentarily bound to the polyT. This mRNA can be, for example, a mRNA available from lysis of a cell, which has been injected into the flow path system, at a predetermined location of the flow path system. It is to be noted that the term “poly” means a nucleic acid molecule having a base sequence formed of two or more bases polymerized together and the term “polyT” means a nucleic acid molecule having a base sequence formed of two or more thymine bases (T). The term “polyAtail” means an adenylic acid residue (ATP chain) added to the 3′ end of a mRNA.

Any nucleic acid can be adopted as the probe nucleic acid, insofar as it is equipped with a function to permit providing information for the detection of target hybridization. Illustrative can be a nucleic acid labeled with a fluorescent substance or a nucleic acid labeled with a radioactive substance.

In the flow path system according to the present invention, a feeding-promoting section may be arranged in a region on a downstream side of the reaction section such that the feeding-promoting section can contribute to a speed-up in the feeding of each solution. This feeding-promoting section can adopt, for example, a construction with perfusion chromatography particles packed therein. It is also possible to adopt a construction of the form that the reaction section and the subsequent feeding-promoting section are repeatedly arranged in two or more combinations. At this feeding-promoting section, substances which have flowed past the reaction section, for example, the probe nucleic acid which has not taken part in the hybridization may be trapped along with any harmful substance or substances.

The present invention also provides a hybridization detecting system including a flow path system of such a construction as described above and a detection unit for detecting hybridization between the target nucleic acid, which exists in the reaction section of the flow path system, and a probe nucleic acid fed into the reaction section.

No particular limitation is imposed on the detection unit in the detecting system, insofar as it is equipped with a detection unit of a construction that can capture information given off from the probe nucleic acid. It is possible, for example, to adopt a detection unit which is equipped at least with an excitation-light irradiating sub-unit for exciting a fluorescent substance labeled on the probe nucleic acid and a photodetector sub-unit for capturing excited fluorescence available from the reaction section.

According to the present invention, the efficiency of reaction is good and the speed-up in the detection of hybridization can be achieved, because it is constructed to allow the hybridization to proceed within a very small space of the flow path system. The arrangement of the feeding-promoting section in the subsequent stage of the reaction section makes it possible to achieve a speed-up in the feeding of a solution. Further, the beads in the reaction section can be repeatedly used by washing them. The present invention can, therefore, be used as a hybridization detecting technique. More specifically, the present invention can be used as a hybridization detecting technique, which assures a good reaction efficiency and can perform the detection of hybridization in a short time.

The above and other objects, features and advantages of the present invention will become apparent from the following description and the appended claims, taken in conjunction with the accompanying drawings in which like parts or elements denoted by like reference symbols.

### BRIEF DESCRIPTION OF THE DRAWINGS

FIG. 1 is a schematic diagram illustrating a flow path system according to a first embodiment of the present invention;

FIG. 2 is a view schematically illustrating the construction of substances on the surface of one of beads packed in a reaction section of the flow path system;

FIG. 3 is a view schematically illustrating a state of hybridization of a probe nucleic acid with a single-stranded segment of a target nucleic acid held on the bead;

FIG. 4 is a schematic diagram showing an illustrative assay method making use of the flow path system according to the first embodiment of the present invention;

FIG. 5 is a schematic diagram depicting a flow path system according to a second embodiment of the present invention;

FIG. 6 is a simplified diagram illustrating a preferred example of a detection unit in a hybridization detecting system making use of the flow path system according to the first or second embodiment of the present invention;

FIG. 7 is a graph as a substitute for drawing, which shows the measurement results of fluorescence intensities as measured at a reaction section (a section packed with oligodT beads) in individual steps of an experiment in Example 2;

FIG. 8 is a graph as a substitute for drawing, which illustrates the results of quantities of fluorescence, which were emitted by the formation of hybrids and were determined based on the data of fluorescence intensities shown in FIG. 7, as corrected by the absorbance of a fluorescent substance Cy3 bound to an oligonucleotide;

FIG. 9 is a graph as a substitute for drawing, which illustrates the results of an experiment in Example 3;

FIG. 10 is a graph as a substitute for drawing, which is a plot of the intensities of fluorescence in FIG. 9 against the corresponding concentrations of a target nucleic acid; and

FIG. 11 is a graph as a substitute for drawing, which shows the results of an experiment (a fluorescence observation experiment at a “feeding-promoting section” in a flow path system device) in Example 4.

### DETAILED DESCRIPTION OF THE PREFERRED EMBODIMENTS

With reference to the accompanying drawings, a description will be made about preferred embodiments for carrying out the present invention. It is, however, to be noted that the individual embodiments shown in the accompanying drawings merely illustrate certain representative examples of systems and methods according to the present invention and that the claims of the present invention shall not be interpreted in any limiting manner based on the embodiments.

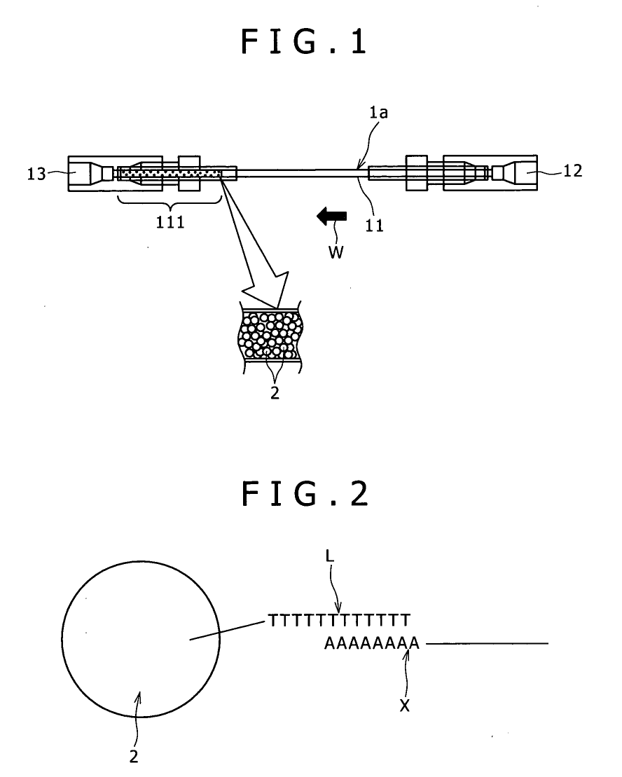

Referring first to FIG. 1, the flow path system according to the first embodiment of the present invention will be described.

Roughly dividing the flow path system indicated at sign 1a in FIG. 1, it is constructed of a capillary 11 of about 500 μm in diameter for serving as a flow path for a sample solution, an inlet port 12 formed at an end of the capillary 11, and an outlet port 13 formed on an opposite end of the capillary 11. It is to be noted that an arrow W shown in FIG. 1 indicates a flowing direction of the sample solution.

The illustrated flow path system 1a is provided at least with a reaction section 111, which is arranged inside the capillary 11 at a location close to the inlet port 12 and is packed with a number of beads 2 of minute particle sizes. This reaction section 111 functions as a site (region) where a desired interaction such as hybridization is allowed to proceed.

With reference to FIG. 2, a description will next be made about the construction of substances on the surface of one of beads 2 packed in the reaction section 111 of the flow path system 1a.

The bead 2 is a minute microbead formed of a material such as polystyrene, and its surface is provided with a construction suited for chemically bonding thereto an end of a linkers L, said end having a polyT sequence.

This bead 2 is provided with the linker L, which is, for example, an oligonucleic acid bonded to the surface of the bead 2 via an avidin-biotin bond or by a coupling reaction (e.g., diazocoupling reaction). The bead 2 also holds a target nucleic acid X, which is complementarily bound at a polyA site thereof to the segment of the polyT base sequence in the linker L based on a polyA selection process. It is to be noted that one of preferred examples of the target nucleic acid X is a mRNA having a polyAtail segment.

In the reaction section 11, the target nucleic acid X is standing by in the state that it is held on the bead 2 via the linker L. The target nucleic acid X plays a role to allow hybridization to proceed with a complementary probe nucleic acid Y in a sample solution to be fed into the reaction section 11.

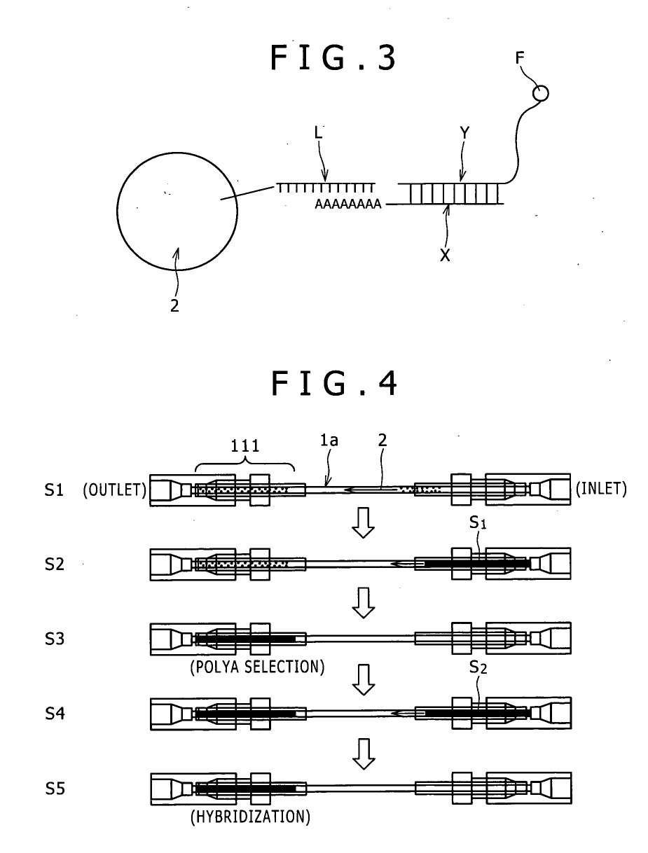

Referring next to FIG. 3, a probe nucleic acid Y has hybridized with a single-stranded segment of the target nucleic acid X held on the bead 2, and therefore, a double-stranded chain has been formed.

As shown in FIG. 3, the advance labeling of the probe nucleic acid Y with a fluorescent substance F or a radioactive substance (not shown), which can be used for the detection of hybridization, makes it possible to detect the hybridization by capturing optical information or radiation information available from the labeling substance.

Such hybridization assay can be used, for example, in a test or the like to determine whether or not a probe nucleic acid Y having a base sequence relevant to a known causative gene for the development of an epidemic disease hybridizes with a mRNA extracted from cells of a subject and held beforehand on beads 2 (via a linker L).

The above assay can be conducted, for example, based on the method illustrated in the flow diagram of FIG. 4. Described specifically, a first step is conducted by packing beads 2 with the linker L immobilized thereon toward the predetermined location in the capillary 11, which makes up the flow path system 1a, and hence, forming the column-shape reaction section 111 (step S1 in FIG. 4).

Toward the reaction section 111 packed with the beads 2, a first sample solution S₁ with the target nucleic acid X contained therein is next fed (step S2 in FIG. 4), and further, the polyA sequence in the target nucleic acid X is caused to hybridize with the polyT segment in the linker L immobilized on the beads 2 (polyA selection process; step S3 in FIG. 4).

A second sample solution S₂ with the probe nucleic acid Y contained therein is then fed toward the reaction section 111 (step S4 in FIG. 4), and at a predetermined temperature, under predetermined pH condition and for a predetermined time, hybridization is allowed to proceed between the target nucleic acid X and the probe nucleic acid Y (step S5 in FIG. 4). Based on information available from the probe nucleic acid Y (for example, excited fluorescence information from the labeling fluorescent substance), the hybridization is detected.

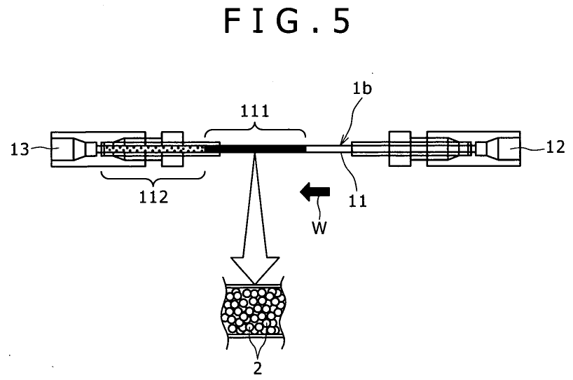

Referring next to FIG. 5, the flow path system according to the second embodiment of the present invention will be described.

The flow path system 1b shown as the second embodiment in FIG. 5 is characterized in that a feeding-promoting section 112 is arranged on a downstream side of the reaction section 111 (in other words, on the side of the outlet port 13). Described specifically, this flow path system 1b is provided with the inlet port 12, the reaction section 111, the feeding-promoting section 112 and the outlet port 13 arranged in this order from the upstream side.

The feeding-promoting section 112 plays a role to promote the feeding speeds of the sample solutions S₁, S₂, a buffer solution and the like to be fed toward the reaction section 111, and can be suitably formed by packing perfusion chromatography particles.

The perfusion chromatography particles are typically equipped with both of large pores called “through pores” and small pores called “diffusive pores”. Owing to these two types of pores, molecules dissolved in a buffer solution are allowed to pass through the through pores and are then brought to every corners of the diffusive pores. A large area of contact is assured between these molecules and the functional groups of the surfaces of the beads so that the distance between the flow of the buffer solution and the functional groups becomes very small (1 μm or less) irrespective of the particle size of the beads. As a consequence, each solution can be fed at a high rate under a low pressure.

At capillary portions adjacent the inlet port 12 and the outlet port 13, various members such as nuts are arranged for the connection with external pumps or the like. The arrangement of the reaction section 111 in such capillary portions, therefore, makes it difficult to perform observations and measurements. The arrangement of the feeding-promoting section 112 on the downward side of the reaction section 111, however, makes it possible to arrange the reaction section 111, which is subjected to a detection or measurement, near a central pat of the flow path system 1b, thereby bringing about a merit in that the detection or measurement is facilitated.

Further, the selection of the type of the perfusion chromatography particles makes it possible to adsorb and trap any surplus substance and harmful substance (for example, radioactive substance) to avoid their discharge to the outside. This had brought about another merit in that such surplus substance and harmful substance can be discarded concurrently with the disposal of the flow path system 1b.

The above-described flow path system 1a or 1b can be used singly, or as an alternative, it is also possible to adopt a multiple flow system constructed of plurality of such flow path systems combined together (not shown). For example, by using a single inlet portion 12 in common and enabling to concurrently feed the same sample solution to the plurality of flow path systems 1a(1b) and further, by holding target nucleic acids X of different kinds within the reaction sections 111 of the respective flow path systems, a comprehensive detection of respective hybridizations can be performed at the same time.

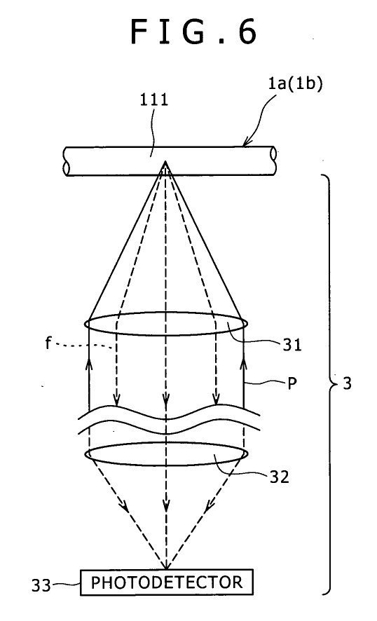

With reference to FIG. 6, a description will next be made about the preferred example of the detection unit in the hybridization detecting system making use of the flow path system 1a or 1b.

The detection unit 3 indicated by numeral 3 in FIG. 6 is equipped with a typical construction for the fluorometric detection of hybridization. The probe nucleic acid Y which exists in a hybridized state in the reaction section 111 has been labeled beforehand with a fluorescent substance.

Toward the reaction section 111 of the flow path system 1a(1b), fluorescent excitation light P of a predetermined wavelength is emitted from an unillustrated light source. On its way toward the reaction section 111, the fluorescent excitation light P is converted into parallel rays. Through a lens 31 arranged in the proximity of the reaction section 111, these parallel rays are then condensed and irradiated toward the reaction section 111.

Fluorescence f, which have been exited in the reaction section 111 as a result of the irradiation of the parallel rays, is converted into parallel rays through the lens 31, is condensed through a lens 32 arranged on a rear side, and is then detected by a photodetector 33 arranged on a still rear side to measure the intensity of the fluorescence. It is to be noted that the detection unit useful in the present invention is not limited to the above-described fluorescence detection unit and that depending on the type of information available from the probe nucleic acid Y, any construction can be adopted insofar as it can detect the information.

### EXAMPLE 1

<Example Relating to the Fabrication of a Flow Path System Device>

First of all, a fused silica capillary tube of 0.53 mm in inner diameter, 0.68 mm in outer diameter and 6 cm in length (product of GL Sciences Inc.) was provided as a capillary for constructing a flow path system.

An outlet port with a filter of 1 μm in pore size fitted therein and a filter-free inlet port were then attached to a downstream-side end portion and an upstream-side end portion, as viewed in the direction of feeding of each solution, of the fused silica capillary tube by means of tubing sleeves, ferrules and nuts, respectively. In addition, a fill port corresponding to “RHEODYNE” syringe (product of RHEODYNE LLC) loading injectors is fitted on the upstream-side inlet port, and a Luer-lock needle was fitted in the downstream-side outlet port.

<Preparation of Perfusion Chromatography Particles>

As perfusion chromatography particles, “POROS 20 R1” (trade name, product of Applied Biosystems Japan Ltd.) was used. “POROS 20 R1” was dispersed in a 10% ethanol solution to prepare a particle dispersion (hereinafter called “the POROS solution”).

<Preparation of Oligonucleotide-Coupled Microbeads>

A 5′biotinylated oligonucleotide (21 mer) of deoxythymidine was then added to an aqueous solution of “Streptavidin Coated Microsphere plain” (trade name; Polysciences, Inc.) to prepare beads with oligodT immobilized thereon via avidin-biotin bonds (hereinafter referred to as “oligodT beads”).

<Formation of Column (Feeding-Promoting Section and Reaction Section)>

A syringe was attached to the Luer-lock needle in the outlet port of the flow path system device. A “RHEODYNE” syringe (product of RHEODYNE LLC) loading injector with the “POROS” solution drawn therein was fitted to the fill port on the inlet port. A plunger of the syringe attached to the Luer-lock needle was pulled to inject the “POROS particles” as perfusion chromatography particles into the capillary tube.

Subsequently, the “oligodT beads” prepared by the above-described procedure were injected into the capillary tube in a similar manner. As a result, a flow path system device similar to that illustrated in FIG. 5 was formed with a two-stage column structure formed of a feeding-promoting section (a section packed with perfusion chromatography media particle) and a reaction section (a section packed with the oligodT beads).

The Luer-lock needle and fill port were next detached from the flow path system device, and were fixed on a heat plate as a stage in a fluorescence microscope. A capillary with ferrules and nuts attached to respective ends thereof to permit a connection to the inlet port, a capillary with a ferrule and nut attached to only one end thereof, a syringe pump, and an effluent bottle were provided.

Then, the capillary with the ferrules and nuts attached to the respective ends thereof was provided and attached to the upstream-side inlet port of the flow path system device. The ferrule and nut on the opposite end of the capillary were attached to the Luer-lock needle so that the capillary was connected to the syringe set on the syringe pump. To the downstream side of the flow path system device, the capillary with the ferrule and nut attached to only the one end thereof was attached, and the opposite end of the capillary was introduced into the effluent bottle.

### EXAMPLE 2

<Preparation of Oligonucleotide Solution>

An oligonucleotide (hereinafter referred to as “Cy3-oligodG+oligodA”, 42 mer in total) formed of deoxyguanosine (21 mer) labeled at the 5′end thereof with a fluorochrome Cy3 and deoxyadenosine (21 mer) was firstly provided as a target nucleic acid (see Table 1).

TABLE 1

| Target nucleic acid | The number of bases (mer) |
|---|---|
| Cy3-oligodG + oligodA | 42 (21 mer oligodG + 21 mer |
| (5′end [symbol] →3′end) | oligodA) |

Two types of oligonucleotides were next provided as probe nucleic acids, one being oligodeoxyguanosine (Cy3-oligodG, 21 mer) labeled at the 5′end thereof with a fluorochrome Cy3 and the other oligodeoxycytidine (Cy3-oligodc, 21 mer) labeled likewise (see Table 2).

TABLE 2

| Type of probe | Constituent nucleic | The number of |
|---|---|---|
| nucleic acid | acid | bases (mer) |
| First probe nucleic | Oligodeoxyguanosine | 21 |
| acid | (Cy3-oligodG) | |
| Second probe | Oligodeoxycytidine | 21 |
| nucleic acid | (Cy3-oligodC) | |

The thus-provided oligonucleotides were separately dissolved at 5 μM concentration in aliquots of a 0.5 M aqueous solution of sodium chloride. It is to be noted that under the condition of 0.5 M sodium chloride, an oligonucleotide is known to form a hybrid by itself (Kazuhiro W. Makabe: “Bioexperiments Illustrated”, volume 4, Trouble-Free Cloning”, published in Japanese, Chapter 1, Paragraph 2, 1997, Shujunsha Co., Ltd., Tokyo)

<Experimental Procedure and Results>

This experiment was conducted using the flow path system device prepared as fabricated above. Temperature control of the flow path system device was performed using the heat plate. The amount and flow rate of each oligonucleotide solution to be fed into the flow path system were set at 200 μL and 50 μL/min, respectively. Observation of fluorescence from Cy3 for the confirmation of hybridization was performed by a microspectrophotometric system (manufactured by Otsuka Electronics Co., Ltd.) connected to the fluorescence microscope.

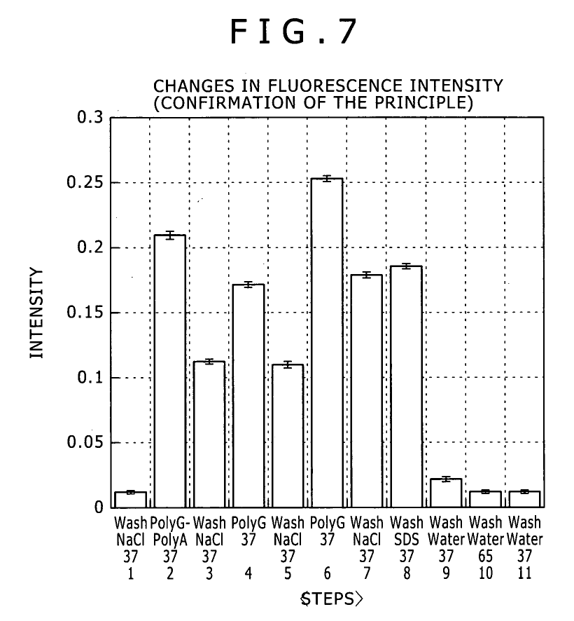

The measurement results of fluorescence intensities measured at the reaction section (the section packed with the oligodT beads) in the respective steps of the experiment are shown in FIG. 7. It is to be noted that in FIG. 7, the steps are plotted in their order along the abscissa and the fluorescence intensities immediately after conducting the respective steps are plotted along the ordinate.

In the experiment, conditioning of the column with a 0.5 M aqueous solution of sodium chloride was firstly performed under the temperature condition of 37° C. before feeding the target nucleic acid (see Table 1) into the device (see the bar corresponding to Step 1 (Wash NaCl 37) in FIG. 7). For the conditioning, the 0.5 M aqueous solution of sodium chloride (800 μL) was fed. At this stage, practically no fluorescence intensity was measured.

“Cy3-oligodG+oligoda”, a target nucleic acid of 42 mer (see Table 1), (200 μL) was next fed into the device (temperature condition: 37° C.). The fluorescence intensity at that time increased to a level higher than 0.2 as indicated by the bar corresponding to Step 2 (PolyG-PolyA 37) in FIG. 7.

While maintaining the device under the temperature condition of 37° C., a 0.5 M aqueous solution of sodium chloride (800 μL) was then fed (see the bar corresponding to Step 3 (Wash NaCl 37) in FIG. 7). At that time, the fluorescence intensity dropped, but a florescence intensity of a value higher than 0.1 was still indicated. It was, therefore, possible to confirm the polyA selection between the oligodT(linker) immobilized on the beads and the target nucleic acid. In other words, the hybridization between the oligodT immobilized on the beads and the 3′oligodA of the target nucleic acid was confirmed.

A sample solution (200 μL) containing Cy3-oligodG as the first probe nucleic acid (see Table 2) was then fed into the device under the temperature condition of 37° C. As a result, the fluorescence intensity increased to about 0.17 (see the bar corresponding to Step 4 (PolyG 37) in FIG. 7).

While maintaining the temperature condition at 37° C., a 0.5 M aqueous solution of sodium chloride (800 μL) was then fed to wash the device. As a result, the fluorescence intensity dropped (see the bar corresponding to Step 5 (Wash NaCl 37) in FIG. 7). The fluorescence intensity at that time was equal to the fluorescence intensity after the first washing step subsequent to the feeding of the target nucleic acid (see FIG. 7 and Table 1). This is considered to indicate that the increase in fluorescence intensity after the feeding of the sample solution containing the first probe nucleic acid Cy3-oligodG was not brought about by the formation of a complete hybrid but was caused by the occurrence of nonspecific adsorption.

The above-provided sample solution (200 μL) which contained the second probe nucleic acid Cy3-oligodC was then fed at the temperature condition of 37° C. into the device. At that time, the fluorescence intensity increased to a level higher than 0.25, and therefore, indicated a value higher than that measured when the first probe nucleic acid Cy3-oligodG was fed as described above (see the bar corresponding to Step 6 (polyC 37) in FIG. 7).

While maintaining the temperature condition at 37° C., a 0.5 M aqueous solution of sodium chloride (800 μL) was subsequently fed for washing the device. As a result, the fluorescence intensity dropped (see the bar corresponding Step 7 (Wash NaCl 37) in FIG. 7). This fluorescence intensity was, however, still higher than the fluorescence intensity subsequent to the second washing (see the bar corresponding to Step 5 in FIG. 7 and compare it with the bar corresponding to Step 7 in the same drawing). It is, therefore, evident that the second probe nucleic acid Cy3-oligodC still remained in the reaction section (the section packed with oligodT beads).

Following Kazuhiro W. Makabe: “Bioexperiments Illustrated”, volume 4, Trouble-Free Cloning”, published in Japanese, Chapter 1, Paragraph 2, 1997, Shujunsha Co., Ltd., Tokyo), washing was conducted under the temperature condition of 37° C. for the sake of double assurance by using a 0.5 M aqueous solution of sodium chloride (800 μL) containing 0.1 DS (sodium dodecylacetate) which is considered to be effective for the avoidance of non-specific adsorption on the beads (see the bar corresponding to Step 8 (Wash SDS 37) in FIG. 7).

At that time, no substantial change took place in fluorescence intensity. The increase in fluorescence intensity was, therefore, considered to be attributed to the formation of complementary G-C bonds and hybridization between the single-stranded polyG fragment (oligodG fragment) of the target nucleic acid, which is in the state complimentarily bonded at its polyA fragment with the oligodT on the beads, and the second probe nucleic acid Cy3-oligodC. In other words, the hybridization between the target nucleic acid (see Table 1) held on the oligodT beads and the second probe nucleic acid (see Table 2) was confirmed.

While maintaining the temperature condition at 37° C., purified water (800 μL) was then fed. As a result, the fluorescence intensity significantly decreased (see the bar corresponding to Step 9 (Wash Water 37) in FIG. 7). Presumably, the hybrid separated into single-stranded chains as a result of a drop in salt concentration, and the oligonucleotide labeled with the fluorescent substance Cy3 was washed away by the feeding of purified water.

Further, the temperature condition was raised to 65° C. and purified water (800 μL) was fed. As a result, the fluorescence intensity dropped to the level after the first washing step (see the bar corresponding to Step 10 (Wash Water 65) in FIG. 7). That fluorescence intensity level remained unchanged even after the temperature was lowered to 37° C. and purified water (800 μL) was fed (see the bar corresponding to Step 11 (Wash Water 37) in FIG. 7). This is considered to be attributable to the complete separation of the double-stranded chains into single-stranded chains and the subsequent washing-away of the single-stranded chains.

From the above experiment, it has been confirmed that only the complementary oligonucleotide forms the hybrid in the flow path system. In other words, it has been successfully confirmed that the single-stranded segment of the target nucleic acid held on the oligodT beads by the polyA selection hybridizes with the probe nucleic acid having the base sequence complementary to the single-stranded strand.

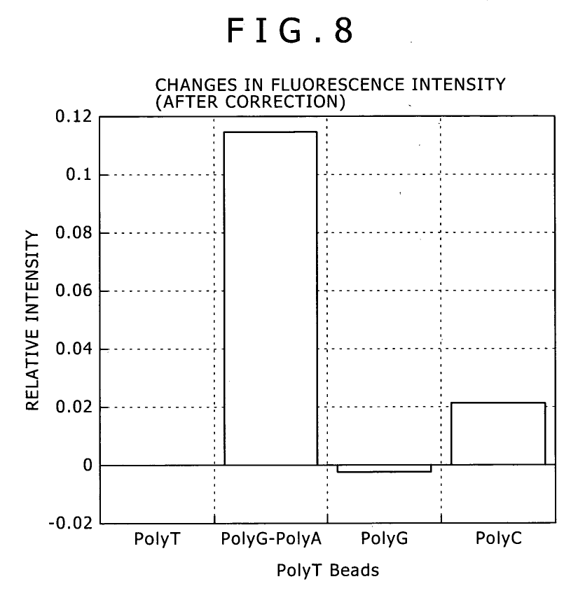

FIG. 8 illustrates the results of quantities of fluorescence, which were emitted by the formation of hybrids and were determined based on the data of fluorescence intensities shown in FIG. 7, as corrected by the absorbance of the fluorescent substance Cy3 bound to the oligonucleotide.

From the results shown in FIG. 8, the corrected fluorescence value by the hybridization between the target nucleic acid and the probe nucleic acid (see the bar corresponding to PolyC in FIG. 8) was about 0.02. On the other hand, the corrected fluorescence value by the hybridization between the linker (the oligodT immobilized on the beads) and the target nucleic acid in accordance with the polyA selection (see the bar corresponding to PolyG-PolyA in FIG. 8) was about 1.0. It has, therefore, been ascertained that the hybridization between the target nucleic acid and the probe nucleic acid is about 20% of the hybridization by the polyA selection.

### EXAMPLE 3

Using the flow path system device fabricated in Example 1, the sensitivity of capture of the target nucleic acid “42 mer Cy3-oligodG+oligodA” (see Table 1) on the oligodT beads by the polyA selection was verified by varying the concentration of the target nucleic acid at four stages in total, that is, 20 μM, 2 μM, 0.2 μM and 0.02 μM.

Conditioning of the column of the reaction section was conducted by feeding a 0.5 M aqueous solution of sodium chloride (800 μL, temperature: 37° C.). The amount of the probe nucleic acid to be fed was set at 200 μL. Washing was conducted by feeding a 0.5M aqueous solution of sodium chloride containing 0.1% SDS (800 μL, temperature: 37° C.). Further, a step of removing any remaining probe nucleic acid from the column of the reaction section was conducted by feeding purified water (800 μL) under the temperature condition of 65° C.

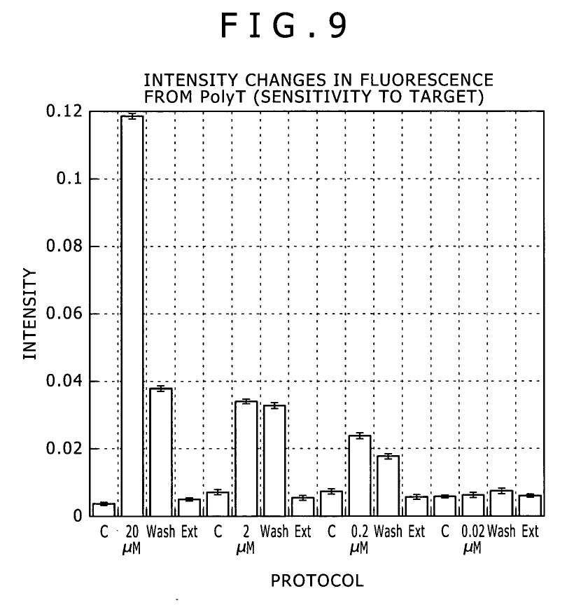

The results of this experiment are shown in FIG. 9. Along the abscissa in FIG. 9, “C” stands for conditioning, “Wash” means washing, and “Ext” indicates the feeding of purified water at 65° C. The concentrations designate the concentrations of the target nucleic acid (Cy3-oligodG+oligoda).

As indicated by the results shown in FIG. 9, it has been successfully confirmed from the fluorescence intensity after washing that it changes depending on the concentration of the target nucleic acid (Cy3-oligodG+oligoda). It was also ascertained that the oligodT beads prepared in this experiment was repeatedly usable.

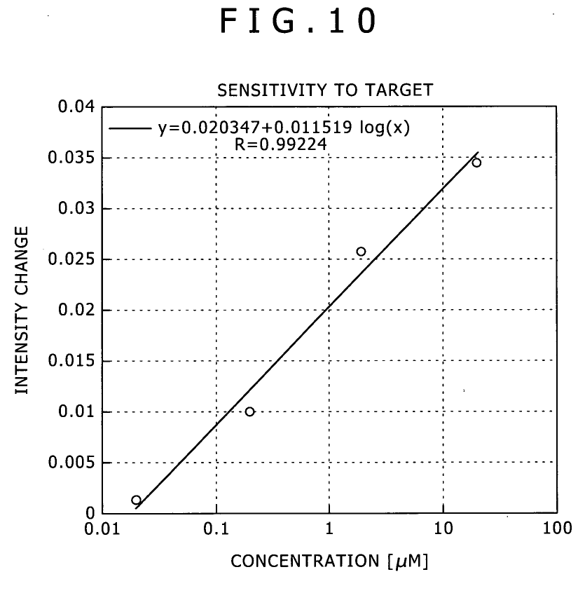

FIG. 10 is a graph as a substitute for drawing, which is a plot of the intensities of fluorescence in FIG. 9 against the corresponding concentrations of the target nucleic acid.

As shown in this FIG. 10, the fluorescence intensity linearly changed in semi-logarithmic scale in the measured concentration range of the target nucleic acid. Taking into consideration that the probe nucleic acid hybridizes with the target nucleic acid as shown in FIG. 8, it has also been found that the target nucleic acid can be quantitated from the fluorescence intensity of the probe nucleic acid because the fluorescence intensity of the probe nucleic acid changes depending on the concentration of the target nucleic acid.

### EXAMPLE 4

During this experiment, the “feeding-promoting section” in the flow path system device was fluorometrically observed.

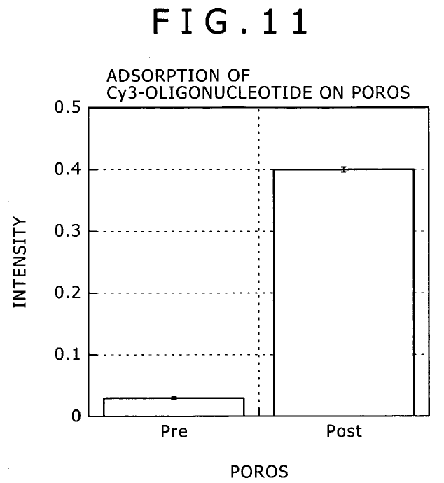

As a result, the fluorescence increased in the course of the experiment. The results are shown in FIG. 11. FIG. 11 illustrates, as a bar graph, fluorescence intensities before the experiment (Pre) and after the experiment (Post). This increase is considered to be attributable to the adsorption of Cy3-oligonucleotide on the perfusion chromatography particles (“POROS” particles).

By selecting perfusion chromatography particles, an adsorbate on the particles can be selected. When a harmful substance (for example, a radioactive substance) is contained in a liquid phase to be fed, it is, therefore, possible to have the harmful substance adsorbed on the perfusion chromatography particles existing in the feeding-promoting section arranged at the rear stage of the reaction section. Accordingly, the harmful substance can be sealed in the device and can be discarded together with the device upon disposal of the device.

The feed rate of each solution through the capillary packed with the beads can hardly be increased to 20 μL/min or higher due to the resistance from the beads. With the flow path system device fabricated above, however, no problem was encountered till 10 mL/min owing to the effects available from the arrangement of the feeding-promoting section. It has, therefore, been successfully verified that the flow rate can be increased by arranging a feeding-promoting section, which is packed with perfusion chromatography particles, in the flow path system.

The present invention can, therefore, be used as a hybridization detecting technique. More specifically, the present invention can be used as a hybridization detecting technique, which assures a good reaction efficiency and can perform the detection of hybridization in a short time.

While a preferred embodiment of the present invention has been described using specific terms, such description is for illustrative purposes only, and it is to be understood that changes and variations may be made without departing from the spirit or scope of the following claims.

What is claimed is:

1. A flow path system comprising:
a capillary provided at a predetermined location thereof with a reaction section packed with beads;
wherein on said beads, a linker having a sequence of bases of the same type is immobilized and a target nucleic acid complimentarily bound to said sequence of said bases of the same type is held.

2. The flow path system according to claim 1, wherein in said reaction section, hybridization is allowed to proceed between a single-stranded segment of said target nucleic acid and a probe nucleic acid fed into said reaction section.

3. The flow path system according to claim 1, wherein said sequence of said bases of the same type in said linker is a polyT, and said target nucleic acid is a mRNA having a polyAtail segment.

4. The flow path system according to claim 3, wherein said mRNA is a mRNA available from lysis of cells, which have been injected into said flow path system, at a predetermined location in said flow path system.

5. The flow path system according to claim 1, wherein said probe nucleic acid is labeled with a fluorescent substance.

6. The flow path system according to claim 1, further comprising
a feeding-promoting section arranged in a region on a downstream side of said reaction section such that said feeding-promoting section can contribute to a speed-up in the feeding of each solution.

7. The flow path system according to claim 1, wherein said feeding-promoting section is packed with perfusion chromatography particles.

8. The flow path system according to claim 6, wherein said reaction section and its subsequent feeding-promoting section are repeatedly arranged in at least two combinations.

9. The flow path system according to claim 6, wherein a fraction of said probe nucleic acid, said fraction having taken no part in said hybridization, is trapped in said feeding-promoting section.

10. A multiple flow path system comprising
a plurality of flow path systems as defined in claim 1.

11. A hybridization detecting system comprising:
a flow path system as defined in claim 1; and
a detection unit for detecting hybridization between said target nucleic acid, which exists in said reaction section of said flow path system, and a probe nucleic acid fed into said reaction section.

12. The hybridization detecting system according to claim 11, wherein said detection unit is provided at least with an excitation-light irradiating sub-unit for exciting a fluorescent substance labeled on the probe nucleic acid and a photodetector sub-unit for capturing excited fluorescence available from said reaction section.
